(19) United States
(12) Patent Application Publication
(10) Pub. No.: US 2011/0144471 A1
(43) Pub. Date: Jun. 16, 2011

(54) FLEXIBLE PROBE STRUCTURE AND METHOD FOR FABRICATING THE SAME

(76) Inventors: Hui-Lin HSU, Taipei (TW); Tri-Rung Yew, Hsinchu (TW); Hsin Chen, Hsinchu (TW); Yung-Chan Chen, Taipei (TW)

(21) Appl. No.: 12/639,605

(22) Filed: Dec. 16, 2009

(51) Int. Cl. A61B 5/0478; H05K 3/10

(52) U.S. Cl. 600/395; 29/846

(57) ABSTRACT

The present invention discloses a flexible probe structure comprises at least one electrode using a CNT layer as the electrode interface. The CNT layer disposed on the electrode surface is processed with an UV-ozone treatment to form a great number of carbon-oxygen functional groups on the surface of CNT. The carbon-oxygen functional groups can greatly reduce the interface impedance of the electrode and the biological tissue fluid. Thereby, the measurement can achieve better quality. The present invention also discloses a method for fabricating a flexible probe structure, which comprises steps: preparing a flexible substrate; forming a conductive layer on the flexible substrate, and defining an electrode, a wire and a metal pad on the conductive layer; forming a CNT layer on the electrode; forming an insulating layer on the conductive layer to insulate the wire from the environment; and processing the CNT layer with an UV-ozone treatment.

### FIELD OF THE INVENTION

The present invention relates to a flexible probe structure, particularly to a flexible probe structure using a carbon nanotube as the electrode interface. The present invention also relates to a method for fabricating the same flexible probe structure.

### BACKGROUND OF THE INVENTION

In neural physiology, a neural probe is usually used to stimulate and measure neural cells to study the physiological operation statuses of nerves. When neural cells convert or transmit electric signals via the differences of the electric potentials thereof, the electrode of a neural probe can measure the intracellular or extracellular neural signals and then receive and transmit the nerve impulses created by the electric potential differences. The study of neural physiology can improve the understanding of neural diseases, such as the Alzheimer's disease, Parkinson disease, dystonia, and chronic pain.

In detecting extracellular neural signals, the neural electrode has to closely contact neural cells and electrically stimulates/detects the neural cells in a capacitive coupling way. The efficiency of the abovementioned capacitive coupling correlates with the selectivity, sensitivity, charge transfer characteristics, long-term chemical stability, and interfacial impedance between the neural electrode and the cell tissue.

The silicon-based neural probe can be fabricated with the MEMS (Micro-Electro-Mechanical System) technology and thus can massively replace the traditional metallic probe. However, the silicon-based probe is very hard and unlikely to bend or deform. When the testee moves, the silicon-based probe is likely to harm the tissue and cause inflammation, or even the original test point is displaced and the probe is detached. Therefore, the silicon-based probe is hard to satisfy the requirement of long-term implantation or real-time measurement. In Journal of Micromechanics and Microengineering, vol. 14, pp. 104-107, 2004, the research team of Takeuchi proposed a “3D Flexible Multichannel Neural Probe Array” to overcome the problem that the silicon-based probe harms biological tissues.

CNT (carbon nanotube), which was found by S. Iijima in 1991, has a superior electrical conductivity because of its special structure. Thus, CNT has been widely used in the nanometric electronic elements. CNT has very large surface area (about 700˜1000 m²/g), high electrical conductivity, better physicochemical property, better chemical inertness and better biocompatibility. Therefore, more and more applications use CNT as the neural electrode interface, for example, “Carbon Nanotubes for Neural Interfaces” by David Ricci; “Carbon Nanotube Coating Improves Neuronal Recording” by Edward et al., Nature Nanotech., 2008; “Neural Stimulation with a Carbon Nanotube Microelectrode Array” by Ke Wang et al., Nano Lett., 2006; “Carbon Nanotube Substrate Boost Neuronal Signaling” by Viviana Lovat et al., Nano Lett., 2005; and “Carbon Nanotube Micro-Electrodes for Neuronal Interfacing” by E. Ben-Jacob et al., J. Mater. Chem., 2008.

However, using the CNT as the measurement interface still has room to improve in interface hydrophilicity modification and interface impedance of the biological tissue fluid. Thus, the neural electrode of the present invention integrates a flexible substrate and an electrode interface of the CNT to perform the modification of the surface functionalization to attain higher measurement quality of the neural signals.

### SUMMARY OF THE INVENTION

One objective of the present invention is to provide a flexible probe structure, which can be implanted to a creature to undertake a long-term measurement without causing inflammation of the biological tissue.

Another objective of the present invention is to provide a flexible probe structure, which is exempt from signal attenuation and signal distortion caused by high interface impedance and can obtain higher signal quality.

To achieve the abovementioned objectives, the present invention proposes a flexible probe structure, which is made of a flexible polymeric material with high-biocompatibility and has a CNT (carbon nanotube) electrode interface modified to greatly reduce the interface impedance in measurement. The flexible probe structure of the present invention comprises a base and at least one probe connected to the base. The probe has at least one electrode. The electrode is electrically connected to a metal pad on the base via a wire. The wire is insulated from the environment. The base and the probe are both made of a flexible polymeric material. The electrode has a CNT layer functioning as the electrode interface, and the CNT layer is processed with an UV (ultraviolet ray)-ozone treatment.

The present invention also proposes a method for fabricating a flexible probe structure, which comprises the steps of: preparing a flexible substrate; forming a conductive layer on the flexible substrate and defining an electrode, a wire and a metal pad on the conductive layer; forming a CNT layer on the electrode; forming an insulating layer on the conductive layer to insulate the wire from the environment; and processing the CNT layer with an UV (ultraviolet ray)-ozone treatment.

After being processed with an UV-ozone treatment, the surface of CNT has a great number of carbon-oxygen functional groups. The carbon-oxygen functional groups can greatly reduce the impedance of the interface between the electrode and the biological tissue fluid, whereby is achieved higher measurement quality and increased the adherence of the neural cells to CNT.

Below, the technical contents and embodiments of the present invention will be described in detail in cooperation with the drawings.

### BRIEF DESCRIPTION OF THE DRAWINGS

The embodiments of the present invention will be described in cooperation with the following drawings:

FIG. 1 is a perspective view of the appearance of a flexible probe structure according to one embodiment of the present invention;

FIG. 2 is a diagram showing the relationships of several types of electrode interfaces and the impedances thereof;

FIG. 3A is a diagram schematically showing a first step of a method for fabricating a flexible probe structure according to the present invention;

FIG. 3B is a diagram schematically showing a second step of a method for fabricating a flexible probe structure according to the present invention;

FIG. 3C is a diagram schematically showing a third step of a method for fabricating a flexible probe structure according to the present invention;

FIG. 3D is a diagram schematically showing a fourth step of a method for fabricating a flexible probe structure according to the present invention;

FIG. 4 is a diagram showing the relationship of the intensity and the binding energy of CNT; and

FIG. 5 is a diagram showing the relationship of the impedance and the processing time of the UV-ozone treatment.

### DETAILED DESCRIPTION OF THE PREFERRED EMBODIMENTS

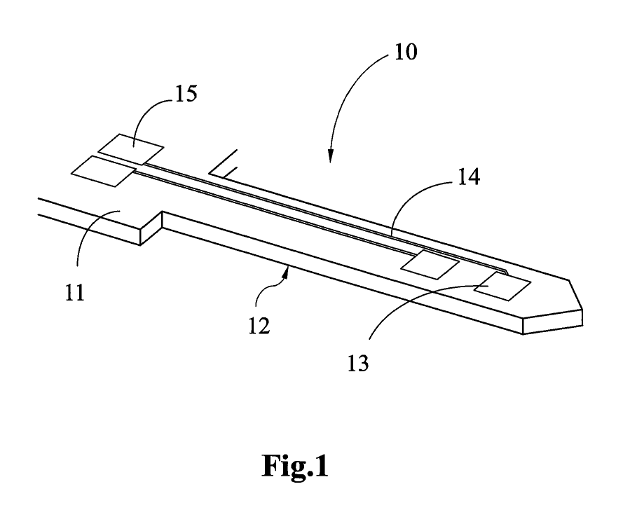

Refer to FIG. 1 a perspective view of the appearance of a flexible probe structure according to one embodiment of the present invention. The present invention proposes a flexible probe structure 10, which comprises a base 11 and at least one probe 12 connected to the base 11. The probe 12 has at least one electrode 13. The electrode 13 is electrically connected to a metal pad 15 on the base 11 via a wire 14. The wire 14 is insulated from the environment. The neural electric signal measured by the electrode 13 is transmitted to the base 11 via the wire 14 and then analyzed by the succeeding devices.

In the flexible probe structure 10, the base 11 and the probe 12 are made of a flexible polymeric material, which is not limited to but may be a material selected from the group consisting of polyimide (PI), poly-para-xylylene (parylene), a thick photoresist SU-8, polydimethylsiloxane (PDMS) and benzocyclobutene (BCB). Thus, the flexible probe structure 10 is bendable and has better biocompatibility and a low-SNR electrophysiological signal. Further, the testee using the flexible probe structure 10 is exempt from the immunological rejection caused by silicon/metal material and thus is free from the inflammation induced by rejection. Therefore, the flexible probe structure 10 can be implanted into a creature to perform long-term measurement. Furthermore, the flexible polymeric material has a lower price and favors mass production.

In the present invention, the electrode 13 has a CNT (carbon nanotube) layer functioning as the measurement interface. The CNT layer is processed with an UV-ozone treatment. In the UV-ozone treatment, the double carbon bonds (C═C) on the outmost layer of CNT are broken by ultraviolet ray, and the carbon atoms thereof react with ozone to form a great number of carbon-oxygen functional groups, such as C—O, C═O, and O—C═O. The carbon-oxygen functional groups form dangling bonds on the surface of CNT and assist in fixing water molecules with the intermolecular bonding therebetween. The carbon-oxygen functional groups provide low-energy absorption sites for water molecules to enhance the reaction capability and charge transferring capability of the electrode 13 and the electrolyte interface mimicking the environment of the biological tissue. Further, the carbon-oxygen functional groups can greatly improve the impedance of the interface of the electrode 13 and increase the adherence of neural cells to CNT, whereby the electrode 13 can attain high-quality and undistorted neural signals. The UV-ozone treatment can improve the wettability of the CNT surface and transform the super-hydrophobic CNT surface into a hydrophilic CNT surface, whereby the CNT can apply to undertake measurement in a biological tissue full of tissue fluid.

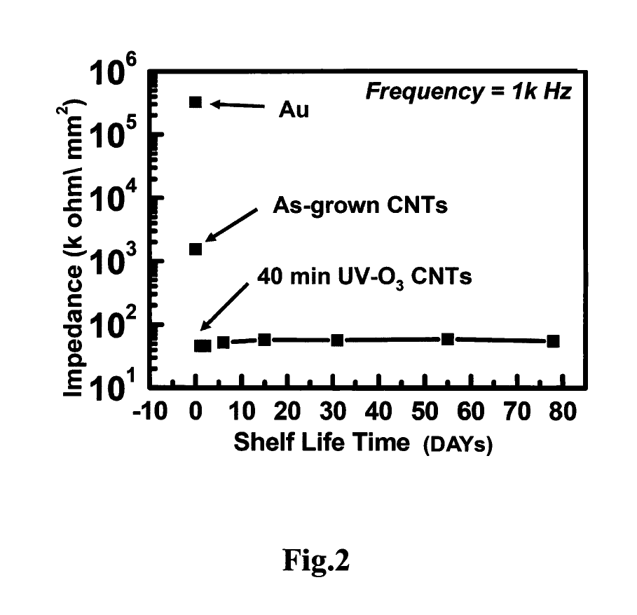

Refer to FIG. 2. In the present invention, an experiment is used to verify that the UV-ozone treatment can greatly decrease the impedance of the interface electrode 13 of CNT, wherein the flexible probe structure 10 is immersed into an electrolyte (such as a 3M KCl solution) mimicking the environment of biological tissue to measure the impedance between the electrode 13 and the electrolyte. In the experiment, the control groups include a traditional gold electrode (designated by Au) and a CNT layer (designated by as-grown CNTs) without an UV-ozone treatment, and an electrode, which has a CNT layer processed with an UV-ozone treatment for 40 minutes (designated by 40 min UV-O₃ CNTs), functions as the experimental group. The impedances of the three groups are compared in FIG. 2. As shown in FIG. 2, the interface impedance of the traditional gold electrode is over 100 times higher than that of the as-grown CNTs. Nevertheless, the impedance of the electrode interface of CNT processed with an UV-ozone treatment is about 100 times lower than that of the as-grown CNTs. Further, the impedance value of the electrode interface of CNT processed with an UV-ozone treatment does not be affected by the using days. Therefore, the flexible probe structure 10 is suitable to a long-term measurement. Furthermore, lower impedance value can reduce the attenuation and distortion of neural signals when the neural signals pass through the electrode. Besides, the UV-ozone treatment can increase the capacitance density of CNT. The flexible probe structure 10 of the present invention can successfully measure the neural signal of the lateral giant neuron of a crayfish. Moreover, the hippocampal neuron cells can be successfully grown on the UV-ozone processed CNT electrode interface, which proves that the UV-ozone treatment can improve the adherence of neural cells to CNT.

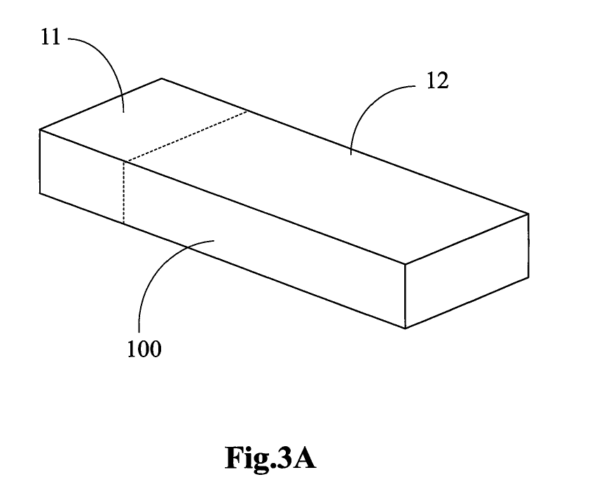

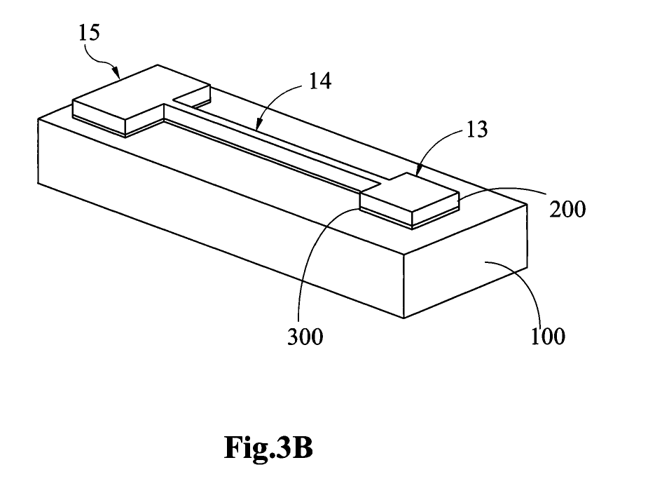

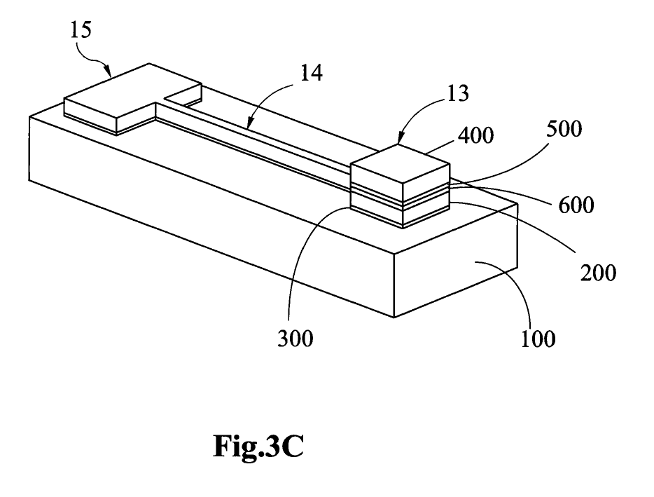

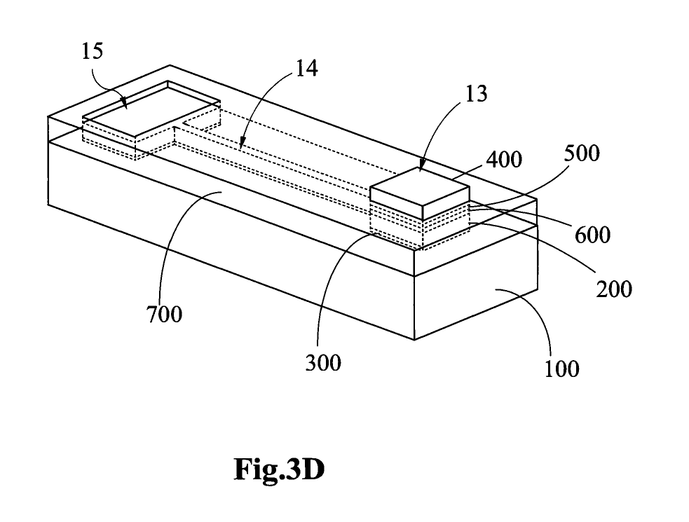

The present invention also proposes a method for fabricating a flexible probe structure 10. Refer to FIGS. 3A-3D. For clear demonstration, the diagrams are not drawn according to the physical proportion. The method of the present invention comprises the following steps:
1. preparing a flexible substrate 100;
2. forming a conductive layer 200 on the flexible substrate 100, and defining an electrode 13, a wire 14 and a metal pad 15 on the conductive layer 200;
3. forming a CNT layer 400 on the electrode 13;
4. forming an insulating layer 700 on the conductive layer 200; and
5. processing the CNT layer 400 with an UV-ozone treatment to form carbon-oxygen functional groups on the outmost layer of the CNT layer 400.

The abovementioned steps will be described in detail below.

As shown in FIG. 3A, a flexible substrate 100 is prepared to function as the main structure of the base 11 and the probe 12 of the flexible probe structure 10. The material of the flexible substrate 100 is not limited to but may be a material selected from a group consisting of polyimide (PI), poly-para-xylylene (parylene), a thick photoresist SU-8, polydimethylsiloxane (PDMS) and benzocyclobutene (BCB). The flexible substrate 100 can be prepared and cut according to the dimensions of the flexible probe structure 10 in advance, for example, according to the appearances, lengths, thicknesses, etc. of the base 11 and the probe 12. Then, the succeeding procedures are undertaken. Alternatively, the abovementioned succeeding procedures can also be undertaken beforehand, and then the flexible substrate 100 is cut to have the desired dimensions. In one embodiment, the flexible substrate 100 is made of polyimide and has a thickness of about 150 μm.

Next, as shown in FIG. 3B, a conductive layer 200 is formed on the flexible substrate 100. A photomask is used to define the predetermined patterns on the flexible substrate 100 so as to form the electrode 13, the wire 14 and the metal pad 15 on different regions of the conductive layer 200. The conductive layer 200 may be made of a metal, such as gold (Au), silver (Ag), aluminum (Al), copper (Cu), platinum (Pt), or an alloy thereof. In one embodiment, the conductive layer 200 is made of gold (Au) and has a thickness of about 150 nm. In one embodiment, an adhesion layer 300 is preformed before the conductive layer 200 is formed on the flexible substrate 100. In one embodiment, the adhesion layer 30 is made of chromium (Cr) and has a thickness of about 2-30 nm.

Next, as shown in FIG. 3C, a CNT layer 400 is formed on the electrode 13. The present invention does not limit the method to form the CNT layer 400. The methods to form the CNT layer 400 include a chemical vapor deposition (CVD) method, a stamp transfer method, a spin-coating method, an ink-jet printing method, a liquid polymer molding method, and a microwave welding method.

In one embodiment, a CVD method is used to synthesize the CNT layer 400 on the electrode 13. Before deposition, a catalytic layer 500 having a thickness of several nanometers to tens of nanometers is formed on the electrode 13 to assist the formation of CNT. The catalytic layer 500 may be made of iron (Fe), cobalt (Co), nickel (Ni), or an alloy thereof. In one embodiment, the catalytic layer 500 is made of nickel (Ni) and has a thickness of about 5 nm; a titanium (Ti) film functioned as a second adhesion layer 600 is adhered to the conductive layer 200, and the catalytic layer 500 is then formed on the second adhesion layer 600. In one embodiment, CNT is synthesized at a temperature of 350-450° C. with a gas flow containing a carbon-source gas (such as methane (CH₄), acetylene (C₂H₂), or ethylene (C₂H₄)), and an inert gas or hydrogen. It should be noted that the abovementioned embodiments are only to exemplify but not to limit the scope of the present invention.

Next, as shown in FIG. 3D, an insulating layer 700 is formed on the conductive layer 200 to insulate the wire 14 from the environment. The insulating layer 700 has a thickness of tens of nanometers to several microns. The insulating layer 700 is made of a high-biocompatibility flexible polymeric material selected from a group consisting of polyimide (PI), poly-para-xylylene (parylene), a thick photoresist SU-8, polydimethylsiloxane (PDMS) and benzocyclobutene (BCB). In one embodiment, the insulating layer 700 is made of parylene and has a thickness of about 1 μm.

When the CNT layer 400 is synthesized with a CVD method, the synthesis is undertaken at a temperature higher than the melting point of the insulating layer 700. In such a case, the CNT layer 400 is formed on the electrode 13 in advance before the formation of the insulating layer 700. In another case, the sequence of forming the CNT layer 400 and the insulating layer 700 may be reversed according to different characteristics and conditions of procedures. For example, the insulating layer 700 can be formed first, and then the CNT layer 400 is formed in a manner that does not damage the insulating layer 700 and the flexible substrate 100 on the electrode 13.

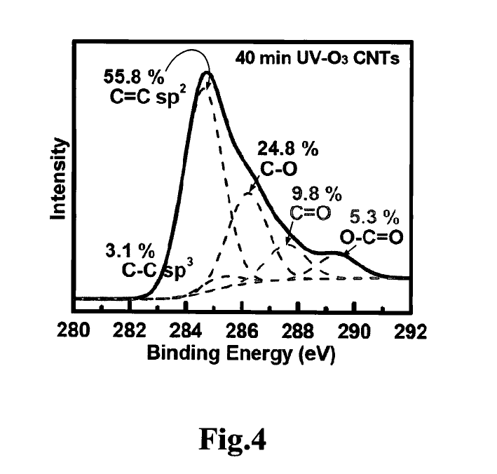

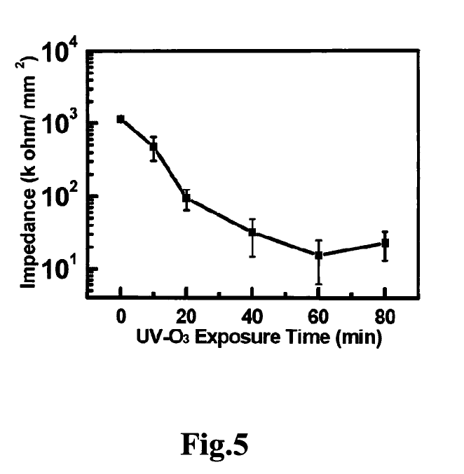

Next, the CNT layer 400 is processed with an UV-ozone treatment. In the UV-ozone treatment, CNT is illuminated with ultraviolet ray in an atmosphere of ozone, whereby the surface of CNT reacts with ozone to form carbon-oxygen functional groups, such as C—O, C═O, and O—C═O. In one embodiment, the ultraviolet ray has an illumination intensity of 25-35 mW/cm² and a wavelength of 254 nm. Refer to FIG. 4 for the relationship of the intensity and the binding energy, the C—C bonds of CNT are converted into different carbon-oxygen functional groups after the UV-ozone treatment. Refer to FIG. 5, the interface impedance between the electrode 13 and the electrolyte decreases with the processing time of the UV-ozone treatment. For example, the CNT layer 400 processed with an UV-ozone treatment for 60 minutes has an impedance less than one hundredth of the original impedance.

Because of adopting a flexible substrate, the flexible probe structure 10 of the present invention is easy to fabricate and has a lower cost. Further, the surface modification of CNT promotes the measurement performance of the flexible probe structure 10 of the present invention.

The embodiments described above are only to exemplify the present invention but not to limit the scope of the present invention. Any equivalent modification or variation according to the spirit and technical contents disclosed in the specification and drawings is to be also included within the scope of the present invention.

What is claimed is:

1. A flexible probe structure comprising a base and at least one probe connected to said base, said probe having at least one electrode, wherein each said electrode is electrically connected to a metal pad on said base via a wire, and wherein said base and said probe are made of a flexible polymeric material, and wherein said electrode uses a CNT (carbon nanotube) layer as an electrode interface, and wherein said CNT layer is processed with an UV (ultraviolet ray)-ozone treatment to form carbon-oxygen functional groups on an outmost layer thereof.

2. The flexible probe structure according to claim 1, wherein said base and said probe are made of a material selected from a group consisting of polyimide, poly-para-xylylene, a thick photoresist SU-8, polydimethylsiloxane and benzocyclobutene.

3. A method for fabricating a flexible probe structure comprising the steps of:
preparing a flexible substrate;
forming a conductive layer on said flexible substrate, and defining an electrode, a wire and a metal pad on said conductive layer;
forming a CNT (carbon nanotube) layer on said electrode;
forming an insulating layer on said conductive layer; and
processing said CNT layer with an UV (ultraviolet ray)-ozone treatment to form carbon-oxygen functional groups on an outmost layer of said CNT layer.

4. The method for fabricating a flexible probe structure according to claim 3, wherein said flexible substrate is made of a material selected from a group consisting of polyimide, poly-para-xylylene, a thick photoresist SU-8, polydimethylsiloxane and benzocyclobutene.

5. The method for fabricating a flexible probe structure according to claim 3, wherein said conductive layer is made of a metallic material selected from a group consisting of gold, silver, aluminum, copper, platinum, and an alloy thereof.

6. The method for fabricating a flexible probe structure according to claim 3, wherein in said UV-ozone treatment, said CNT layer is illuminated with an ultraviolet ray having an illumination intensity of 25-35 mW/cm².

7. The method for fabricating a flexible probe structure according to claim 6, wherein said ultraviolet ray has a wavelength of 254 nm.

8. The method for fabricating a flexible probe structure according to claim 3, wherein said insulating layer is made of a material selected from a group consisting of polyimide, poly-para-xylylene, a thick photoresist SU-8, polydimethylsiloxane and benzocyclobutene.

9. The method for fabricating a flexible probe structure according to claim 3, wherein said CNT layer is formed on said electrode with a chemical vapor deposition method.

10. The method for fabricating a flexible probe structure according to claim 9, wherein said CNT layer is formed on said electrode via a catalytic layer; said catalytic layer is made of a material selected from a group consisting of iron, cobalt, nickel, and an alloy thereof.

11. The method for fabricating a flexible probe structure according to claim 9, wherein said CNT layer is synthesized at a temperature of 350-450° C.

12. A neural electrode using a CNT (carbon nanotube) layer as an electrode interface, wherein said CNT layer is processed with an UV (ultraviolet ray)-ozone treatment.
